(19) United States
(12) Patent Application Publication
(10) Pub. No.: US 2015/0265777 A1
(43) Pub. Date: Sep. 24, 2015

(54) REGIONAL ANESTHESIA SAFETY NEEDLE DEVICE AND METHODS OF USE

(71) Applicant: TELEFLEX MEDICAL INCORPORATED, Research Triangle Park, NC (US)

(72) Inventors: Kenneth W. WHITLEY, Youngsville, NC (US); Sanjib Das ADHIKARY, Hummelstown, PA (US)

(21) Appl. No.: 14/660,302

(22) Filed: Mar. 17, 2015

Related U.S. Application Data
(60) Provisional application No. 61/954,899, filed on Mar. 18, 2014.

(51) Int. Cl. A61M 5/32

(52) U.S. Cl. CPC A61M 5/329

(57) ABSTRACT

A safety needle device includes a housing with a needle and stylet located therein. The needle includes an elongate needle portion with a distal tip. The stylet includes an elongate stylet portion with a distal tip. The safety needle device also includes a compression spring and a sliding member coupled to the needle. A method of navigating the safety needle device toward a point is also provided.

### CROSS REFERENCE TO RELATED APPLICATION

This application claims the benefit of U.S. provisional patent application No. 61/954,899, filed on Mar. 18, 2014, the disclosure of which is hereby incorporated by reference in its entirety.

### FIELD OF THE INVENTION

The present application generally relates to safety needle devices and their methods of use. More particularly, the present application relates to safety needle devices suitable for use in regional anesthesia applications.

### BACKGROUND

Regional anesthesia is a known technique that is commonly performed by clinicians for anesthetizing parts of a patient's body. The anesthetic is typically employed to block nerve supply from the region of interest. By so doing, the patient cannot feel pain in the region of interest during and after surgical procedure.

Patient safety during the administration of anesthesia is a primary concern of clinicians. Generally, regional anesthesia is administered by using a sharp needle. While the sharpness of the needle is important to penetrate the skin or tissue, the clinician must be careful not to accidently insert the needle to an undesirable location beneath the skin's surface. Lack of precision, coupled with variable experience levels, may possibly result in the needle puncturing a nerve residing under the skin or tissue, or entering an unintended area of the body.

Tuohy needles have been employed in the field for many years by clinicians. While Tuohy needles are configured to be sharp enough to puncture a patient's skin or tissue, their main drawback is a reduction in efficiency during administering anesthesia. Namely, the Tuohy needle has a curved distal tip to reduce its sharpness but it is still sharp enough to puncture nerves and unintended structures. Moreover, the Tuohy needle may run the risk of being placed in an undesirable location beneath the skin or tissue resulting in unforeseen complications. For example, if the anesthetic is administered outside of the fascia plane, a delay in blocking the nerve is incurred which may push back surgery. Also, the sensation of pain returns sooner if the anesthetic is not administered directly onto the nerve. Further, overshooting the location for providing anesthesia may possibly result in the puncture of the pleura and introduction in the pleural cavity.

Veress needles have also been employed in the field for many years. Veress needles are commonly used for laparoscopic surgical procedures. The Veress needle includes an outer needle cannula having a shaft with a sharp distal tip for puncturing through skin or tissue. Housed inside the needle cannula is a spring-loaded inner stylet having a dull distal end protruding past the sharp distal tip of the needle shaft. In operation, when pressure is applied to the blunt distal tip of the stylet, and upon contact with skin or tissue, the blunt distal tip is forced back, exposing the sharp tip of the needle. Further insertion of the Veress needle into a body cavity may cause the spring-loaded inner stylet to translate in the axial direction and therefore relieve pressure on the blunt stylet spring allowing it to spring forward. By so doing, the distal tip of the stylet extends past the sharp distal tip of the outer needle, preventing damage to adjacent tissue.

What is desired in the art is a device that precisely punctures the skin of a patient for delivering anesthesia to a region of interest.

Also desired in the art is a device that efficiently punctures skin and delivers anesthesia to a region of interest.

Also desired in the art is a device and method for eliminating or reducing the possibility of puncturing a nerve during administration of anesthesia inside the body.

What is also desired in the art is a method of administering anesthesia to a nerve while eliminating or reducing the possibility of puncturing a nerve.

### SUMMARY OF THE INVENTION

The foregoing needs are met, to a great extent, by the present invention, by a safety needle device and method thereof, employed for safely and efficiently administering anesthesia.

One aspect of the present application advantageously provides a safety needle device including a housing. The housing includes an outer wall, an inner wall, and a proximal and distal end extending along a longitudinal axis of the housing. The safety needle device also includes a needle located in the housing. The needle has an elongate needle portion with a distal tip. The distal tip extends distally from the distal end of the housing. The safety needle device also includes a stylet fixed to the inner wall of the housing. The stylet includes an elongate cannula portion with a blunt distal tip. The device also includes a biasing member coupled to the housing and the needle. Further, the safety needle device includes a sliding member coupled to the needle configured to move the needle between an extended position and a retracted position. Namely, the extended position is a state in which the distal tip of the needle extends beyond the distal tip of the stylet. The retracted position is a state in which the distal tip of the stylet extends beyond the distal tip of the needle. Further, the biasing member acts to bias the needle towards the retracted position.

Another aspect of the present invention advantageously provides a method of improving navigation of a safety needle device toward a point. In one embodiment, the point is a nerve. The method includes a step of providing the safety needle device including an elongate needle portion having a distal tip and an elongate stylet portion having a blunt distal tip. The method further includes a step of inserting the distal tip of the needle through a surface toward the point. Yet another step includes moving the elongate needle portion after the inserting step. The elongate needle portion moves from an extended position where its distal tip extends beyond the distal tip of the stylet to a retracted position where the distal tip of the stylet extends beyond the distal tip of the needle. Further, the safety needle device is advanced while in the retracted position toward the point.

There has thus been outlined, rather broadly, certain embodiments of the invention in order that the detailed description thereof may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, the phraseology and terminology employed herein, as well as the Abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. Therefore, the claims shall be regarded as including such equivalent constructions insofar as they do not depart from the spirit and scope of the present invention.

### BRIEF DESCRIPTION OF THE DRAWINGS

FIG. 1 illustrates a schematic view of a safety needle device in an extended position according to an embodiment of the present invention.

FIG. 2 illustrates a schematic view of a safety needle device in a retracted position according to an embodiment of the present invention.

FIG. 3 illustrates the extended position of the safety needle device wherein a distal tip of the needle extends beyond a distal tip of the stylet according to an embodiment of the present invention.

FIG. 4 illustrates a retracted position of the safety needle device wherein the distal tip of the stylet extends beyond the distal tip of the needle according to an embodiment of the present invention.

FIG. 5 illustrates a cross-sectional view of the safety needle device shown in FIG. 1.

FIG. 6 illustrates a cross-sectional view of the safety needle device shown in FIG. 2.

FIG. 7 illustrates a schematic view of the safety needle device according to another embodiment of the present invention.

FIG. 8 illustrates a cross-sectional view of the safety needle device shown in FIG. 7.

### DETAILED DESCRIPTION

The invention will now be described with reference to the illustrated figures, in which like reference numerals refer to like parts throughout. Moreover, reference elements having the same last two digits are intended to reference similar elements. The drawings should not be construed as limiting the invention. The drawings are intended only to be illustrative.

Reference in this specification to “one embodiment,” “an embodiment,” “one or more embodiments, or the like means that a particular feature, structure, or characteristic described in connection with the embodiment is included in at least one embodiment of the disclosure. Moreover, the term “embodiment” in various places in the specification is not necessarily referring to the same embodiment. That is, various features are described which may be exhibited by some embodiments and not by the other.

Clinicians operating safety needle devices for administering anesthesia generally expect two things—safety and efficiency. The safety needle device described in this application is designed to meet and exceed expectations of the clinician. Moreover, the safety needle device is user-friendly and ergonomic thus increasing its efficiency during administering anesthesia to a patient. Further, the safety needle device is designed and manufactured such that it is cost-effective for single or multiple applications while achieving the demands of the clinician.

In one embodiment, the safety needle device is configured to safely puncture skin or tissue of a patient and thereafter be controllably restrained by a user in an efficient manner. For example, the needle portion may be displaced distally by a user such that a sharp distal tip of a needle is exposed beyond the blunt distal tip of a blunt stylet. The device is in an extended position to be inserted through a substrate, such as for example, skin. After exposure, the needle is displaced proximally by the user to a retracted position such that the blunt distal tip of the stylet is exposed beyond the sharp distal tip of the needle. The user through controlled actions, and assistance via compressive forces in the needle assembly, therefore is able to dictate whether the sharp distal tip remains exposed beyond the blunt distal tip for insertion through skin or subsequent insertion through tissue.

For example, in one embodiment, the user is able to maintain the needle assembly in an extended position by forcibly applying pressure on a sliding lever coupled to a needle. That is, the needle generally remains in a passive state whereby the blunt distal tip of the stylet is extended beyond the distal tip of the needle. Distally applied force from the proximal end of the housing assembly causes axial displacement of the needle. Continuous force, beyond a specific, predetermined amount, retains the needle in the extended position. By so doing, the likelihood of puncturing a point, such as a nerve, is significantly reduced and/or eliminated due to controlled displacement by a user.

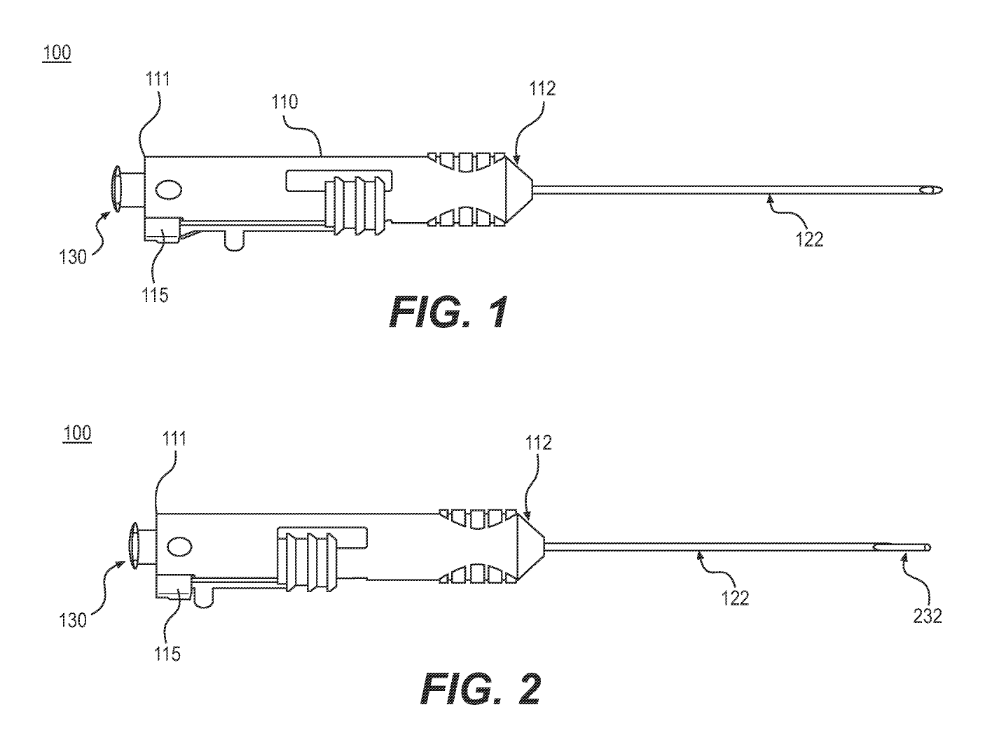

Alternatively, the needle assembly may include a locking mechanism for restricting movement in extended and retracted positions of the needle. Thus, continuous force applied by a user is not required in the extended and retracted positions. FIG. 1, for example, illustrates a side view of the safety needle device 100. The safety needle device 100 can be made in many different sizes. For example, the device 100 can be constructed in small, medium and large sizes in order to accommodate the hands of the clinician operating the device 100. Moreover, the device 100 can be further individualized in view of the area that needs to be anesthetized. In other, words, the device 100 may depend upon the size of the patient.

The safety needle 100 includes a housing 110 formed of a plurality of shapes including but not limited to tubular and rectangular. Preferably, the housing is formed via an injection molding technique. In one embodiment, the housing 110 is tubular including an outer wall and an inner wall. The outer wall may be substantially smooth or alternatively, may include grooved edges which assist the user during insertion through skin or tissue. The inner wall may be defined by a hollow area with a circumference capable of accommodating additional components of the safety needle device 100. Moreover, the housing 110 may formed of any material including, for example, plastic and metal. The material may be selected in view of cost and structural rigidity considerations.

As further shown in FIG. 1, the housing 110 includes a proximal end 111 and a distal end 112 extending along a longitudinal axis. In one embodiment, the proximal end 111 includes an opening. In another embodiment, the distal end 112 includes an opening. Preferably, the opening is configured to accommodate a component of the device 100.

In a subsequent embodiment, the housing 110 further includes a protrusion member 115. In one embodiment, the protrusion member 115 is separably attached to the housing 110. In an alternative embodiment, the protrusion member 115 and housing 110 are formed as a single, unitary structure. As will be discussed in more detail below, the protrusion member 115 is configured to help prevent the device 100 from puncturing a nerve.

Specifically, the protrusion member 115 may be formed of any shape and is customizable to meet the function of the device 100. In one embodiment, the protrusion member 115 generally extends perpendicular to an outer wall of the housing 110. The protrusion member 115 is also generally located adjacent to the proximal end 111 of the housing 110. In yet another embodiment, the protrusion member 115 includes a slot formed therein. The slot is formed in the protrusion member 115 in a manner to serve as a locking member. The slot will be discussed in more detail below in reference to the locking mechanism.

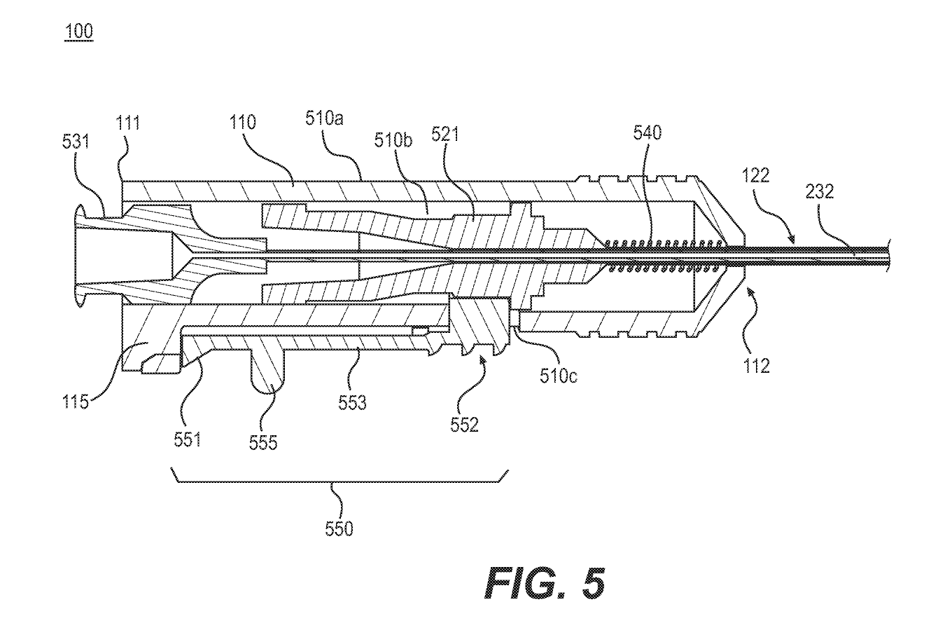

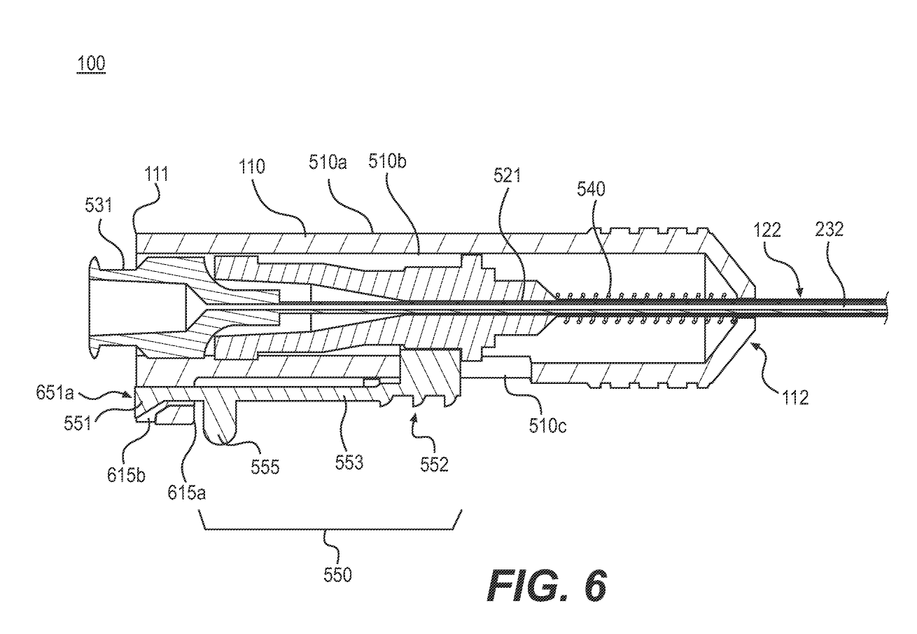

As shown in FIGS. 1 and 2, the safety needle device 100 also includes a needle assembly including an elongate portion 122. Any material generally known in the art to the skilled person to make a needle may be employed herein. As further shown in FIGS. 5 and 6, the needle assembly includes a needle hub 521 configured to fit within an inner wall 510b of the housing 110. In an exemplary embodiment, the needle hub 521 is entirely located within the housing 110. In yet another embodiment, the needle hub 521 may be configured so as to be movable within the housing 110. Namely, the needle hub 521 may be movable from a first position that is located adjacent the distal end 112 of the housing 110 to a second position that is located adjacent the proximal end 111 of the housing 110.

As shown in FIGS. 5 and 6, the needle assembly includes an elongate portion 122 extending from one end of the needle hub 521. In one embodiment, the elongate portion 122 extends distally to the distal end 112 of the housing 110. In another embodiment, the elongate portion 122 extends through a distal end 112 of the housing 110 to an external environment. The elongate needle portion 122 of the needle assembly, and preferably, the entire needle assembly, is typically formed from a rigid material such as metal, stainless steel, or a plastic material. Importantly, the elongate portion is substantially rigid enough to puncture the subcutaneous tissue. The elongate portion 122 may generally be hollow through its center. Preferably, the hollow center is capable of accommodating another component of the safety needle device 100. Moreover, the elongate portion 122 includes an inner and outer diameter having a thickness therebetween. The thickness between the inner and outer diameters is variable and depends upon customer demand.

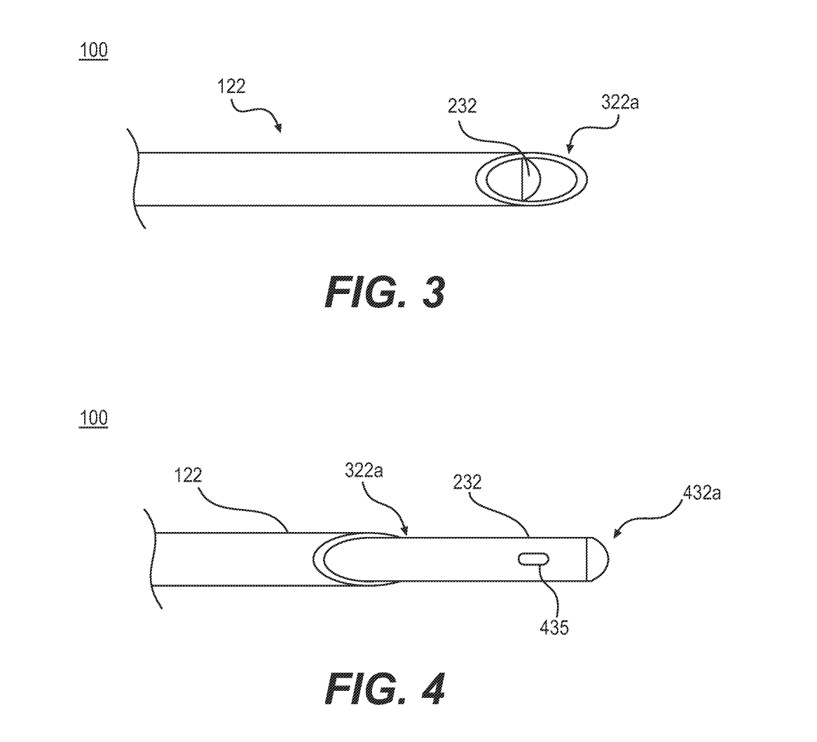

The elongate needle portion 122 further includes a distal end 322a as shown in FIGS. 3 and 4. The distal end is generally beveled from one edge to another edge forming a tip. In an exemplary embodiment, the beveled tip is formed to be sufficiently sharp enough to puncture skin and the surrounding or underlining tissue.

As shown in FIGS. 1 and 2, the safety needle device 100 also includes a stylet 130. The stylet is not limited to a particular shape. The stylet is also not limited to being constructed from a particular material and may include, for example, plastics or metals as generally known by those skilled in the art. As further shown in FIGS. 5 and 6, the stylet includes a stylet hub 531 that is substantially located within an inner wall 510b of the housing 110. In another embodiment, a portion of the stylet hub 531 includes extends through a proximal end 111 of the housing 110. In yet another embodiment, the stylet hub 531 is fixed to an inner wall of the housing 110. Specifically, the stylet hub 531 does not translate or move during movement of the needle hub 521 and/or the elongate portion 122. In a further embodiment, the stylet hub 531 and the elongate stylet portion 232 do not translate during movement of the needle hub 521 and/or the elongate portion 122.

As shown in FIGS. 5 and 6, one end of the stylet hub 531 is coupled to the elongate stylet portion 232. In one embodiment, the elongate stylet portion 232 extends to the distal end 112 of the housing 110. In another embodiment, the elongate stylet portion 232 extends through the distal end 112 of the housing 110.

As further shown in FIG. 5, the elongate stylet portion 232 also extends through the needle hub 521. In a preferred embodiment, the elongate stylet portion 232 extends through the needle hub 521 at all times during operation. In other words, the elongate stylet portion 232 extends at least through the needle hub 521 when it is in a first position, e.g., adjacent the distal end of the housing or the second position, e.g., adjacent the proximal end of the housing.

As illustrated in FIG. 4, the elongate stylet portion 232 includes a distal tip 432a. The distal tip 432a is generally blunt. For example, the blunt tip may be round. The blunt distal tip is intended to be incapable of puncturing skin or tissue. The elongate stylet portion 232 is generally hollow in order to transmit fluids, e.g., anesthetic, there through for purposes of anesthetizing nerves in the surrounding area. Moreover, the outer diameter of the elongate stylet portion 232 is large enough to fit within the inner diameter of the elongate portion 122.

In an embodiment, the elongate stylet portion 232 may further include an opening 435 configured to permit anesthetic to be output therefrom. Specifically, the opening 435 may be formed of any shape including but not limited to a square, rectangle, oval and circle. As illustrated in FIG. 4, the opening 435 is oval-shaped. The opening 435 may be formed on any surface of the elongate portion 432. For example, the opening 435 may be formed on the distal tip 432a. Alternatively, the opening 435 may be formed along a side surface capable of being exposed to a foreign environment, such as for example, tissue, when the needle is moved to a retracted position.

As further illustrated in FIGS. 3 and 4, the elongate stylet portion 232 is disposed within the elongate portion 122 of the needle. In other words, the elongate stylet portion 232 is coaxially nested within a lumen of the elongate portion 122. The elongate stylet portion 232 also may include a blunt tip 432a that is exposed and extended outside of the distal tip 322a of the needle when the needle is in a retracted position. By so doing, the blunt tip effectively prevents the sharpened distal tip from causing trauma to, or puncturing, skin, or other tissue. One the other hand, when the needle is in an extended position, as shown in FIG. 3, the blunt tip is entirely disposed within the needle such that the distal tip 322a is the first part of the safety needle device 100 contacting a substrate, such as for example, skin or tissue. The stylet being maintained within the bevel of the needle prevents coring of the tissue through which a needle is advanced by a clinician.

As illustrated in FIGS. 5 and 6, the safety needle device 100 may also include a biasing member 540. In one embodiment, the biasing member can be a spring. The spring 540 can be located within an inner space defined by the housing 110, such as between the distal end 112 of the housing 110 and one end of the needle hub 521. In one embodiment, the spring 540 is located around a section of the elongate portion of the needle.

In the embodiment shown in FIGS. 5 and 6, the safety needle device 100 includes a locking mechanism including a locking handle 550. The locking handle 550 is coupled to the needle assembly. The locking handle 550 is configured to move the needle hub 521, together with the elongate needle portion 122, between two positions. Specifically, the first position is referred to as the “extended” position. The extended position is the state in which the distal tip of the elongate portion 122 extends beyond the distal tip of the elongate stylet portion 232. The second position is referred to as the “retracted” position. The retracted position is the state in which the distal tip of the elongate stylet portion 232 extends beyond the distal tip of the elongate portion 122. In an exemplary embodiment, at least a section of the elongate portion 232 of the stylet is disposed within an area defined by an inner wall of the elongate portion 122 of the needle. Moreover, the stylet and needle can be locked, i.e., fixed in terms of displacement, relative to each other in both of the extended and retracted positions. By so doing, the needle device provides safety for involuntary punctures.

The locking handle 550 may also be referred to as a sliding section primarily due its ability to axially slide between the extended and retracted positions. In an exemplary embodiment, the locking handle 550 is substantially disposed outside of the housing 110.

In the embodiment illustrated in FIG. 5, the locking handle 550 is substantially arranged along an outer wall 510a of the housing 110. The locking handle 550 is also disposed between the needle hub 521 and a proximal end 111 of the housing 110. In one embodiment, the locking handle 550 includes a terminal portion 551 that is configured to extend toward a proximal end 111 of the housing 110. Moreover, at least one surface of the terminal portion 551 is substantially parallel to an outer wall of the housing.

In yet another embodiment, the locking handle 550 may include, or be coupled to, a sliding member 552 that is fixably coupled to the needle assembly. More preferably, the sliding member 552 is coupled to the needle hub 521 of the needle assembly. In one embodiment, the sliding member 552 includes at least one thread disposed thereon. In a preferred embodiment, the sliding member 552 includes at least two threads disposed thereon. The threads may be equidistantly located from one another. The threads function to provide the clinician with a grip when maneuvering the locking handle. The sliding member 552 is preferably coupled to the needle hub 521 via an opening extending between an outer wall 510a and an inner wall 510b of the housing 110. Alternatively, the sliding member 552 may be shaped as a lever capable of being axially displaced by a user toward a distal end of the housing.

Moreover, the outer wall of the housing may include a recessed area 610b whereby the sliding member 552 is capable of sliding from a first position and a second position. As shown in FIG. 5, the sliding member 552 is in a first position located closer to the distal end 112 of the housing 510. Conversely, FIG. 6 illustrates the sliding member 552 being located in a second position located closer to the proximal end 111 of the housing 110. The recessed area can formed of an appropriate depth such that the connecting end is capable of sliding between the first and second ends.

In a further embodiment, the locking handle 550 further includes a stopping member 555 disposed between the terminal portion 551 and sliding member 552. The function of the stopping member 550 is to restrain the locking handle 550 from moving further along an axial direction with respect to the housing 110 toward a proximal end 111 thereof. The stopping member 555 can be formed in any shape. In a preferred embodiment, the stopping member is a semi-circle.

In yet another embodiment, the stopping member 555 extends perpendicular to a main portion 553 of the locking handle 550. Preferably, the stopping member 555 is disposed along a first surface of the main portion. Preferably, the main portion 553 extends substantially parallel to an outer wall 510a of the housing 110. The main portion 553 is further arranged in a manner wherein a spaced gap is formed between a second surface of the main portion 553 and an outer wall 510a of the housing 110.

According to another aspect of the present invention, there is described a method of operating the safety needle device 100. Namely, FIGS. 5 and 6 illustrate cross sectional views of the safety needle device 100. FIG. 5 depicts the safety needle device 100 in an extended position. Meanwhile, FIG. 6 depicts the safety needle device 100 in a retracted position.

According to FIG. 5, when the safety needle device 100 is oriented in an extended position, the spring 540 is loaded with a compressive force and configured to be in a contracted state between the needle hub 521 and the distal end of the housing 110. The locking handle 550 is positioned in a manner such that the proximal terminal portion 551 is located abutting the protrusion member 115. In an exemplary embodiment, the proximal terminal portion 551 includes a major surface abutting against a first surface of the protrusion member 515. This abutting arrangement prevents the needle from moving in an axial direction toward the retracted position. In use, clinicians will seek to make a puncture through skin when the needle is configured in the extended position as shown in FIG. 5.

In FIG. 6, when the safety needle device 100 is oriented in a retracted position, the spring 540 is in a relaxed state between the needle hub 521 and the distal end 112 of the housing 510. The locking handle 550 is positioned in a manner such that the stopping member 555 is in a second position along the axial direction of the housing 110 whereby it is proximal to the protrusion member 115. The proximal terminal portion 551 of the locking handle 550 is moved via a force such that it engages with the protrusion member 115 in a slot 615b defined by the protrusion member 115. To achieve locking via the locking handle 550, force is generally applied thereon such that the main portion 553 is displaced toward the outer wall 510a of the housing 110 resulting in the gap disposed therebetween to become reduced in area. The force ultimately is translated to the proximal terminal portion 551. More specifically, the proximal terminal portion 551 having a major surface 651a, slides away from a first surface 615a of the protrusion member. Once the major surface 651a clears an edge of first surface 615a, the proximal terminal portion 551 moves or slides in an axial direction toward the proximal end of the housing 110.

In a further embodiment, the proximal terminal portion 551 slides into, and is received in a slot 615b which extends toward a proximal end 111 of the housing 110. Namely, the slot 615b is formed between the first surface 615a of the protrusion member 115 and an outer wall 510a of the housing 110 adjacent the proximal end 111 thereof. The slot 615b may be formed in any shape, however, it must at least be of a sufficient width and length to accommodate the dimensions of the proximal terminal portion 551 and/or at least a section of the main portion 553 of the locking handle 550. In a further embodiment, the proximal terminal portion 551 may extend to the proximal end 111 of the housing 110.

Further, the stopping member 555, as discussed above, should be formed such that its width and/or height is greater than the width and/or height of the slot 615b. By so doing, the locking handle 550 is restrained from moving beyond the protrusion member 115.

As further shown in FIG. 6, the stylet hub 531 includes a portion thereof that is located within an inner circumference of the needle hub 521. As explained above, the stylet hub 531 and elongate stylet portion 532 remain static such that only the needle assembly is configured to move between the extended and retracted positions.

As discussed above, the proximal terminal portion 551 is located between the stopping member 555 and the protrusion member 115. In an exemplary embodiment, the proximal terminal portion 551 includes a major surface 651a which may abut against a first surface 615a of the protrusion member. The abutment prevents the needle assembly from retracting. Clinicians will seek to make a puncture through skin when the needle is oriented in the extended position as shown in either FIG. 1 or 5. Moreover, the extended position of the needle causes the needle hub to be fixed in an area of the housing 110 that is substantially exclusive of an area occupied by the stylet hub 531. That is, while a small portion of the stylet hub 531 may lay within the inner circumference of the needle hub 521, a substantial portion of the stylet hub 531 lies outside the needle hub 521.

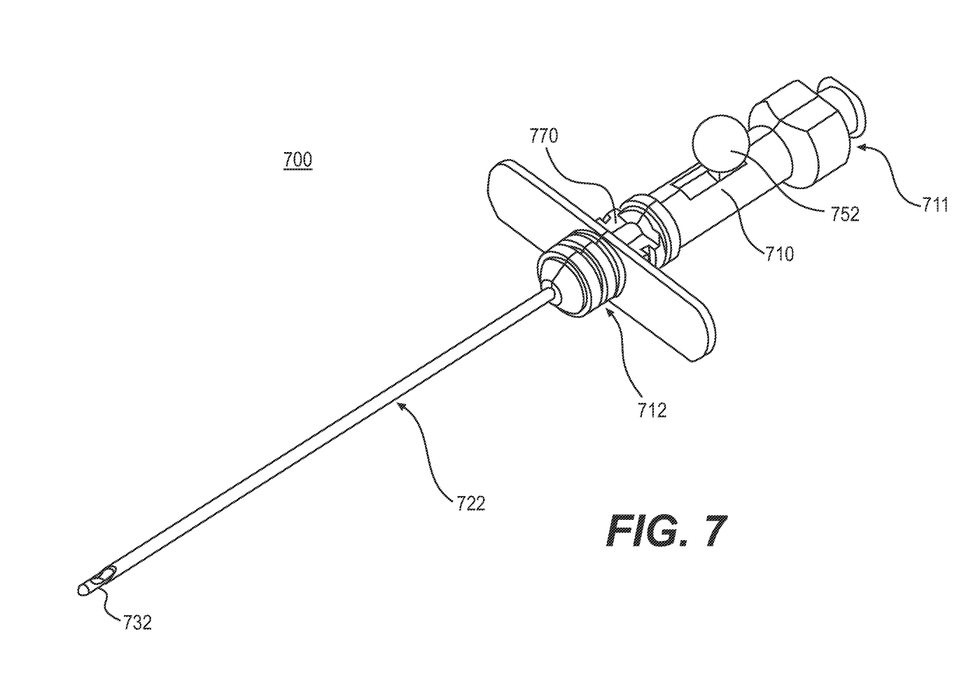

According to another embodiment as illustrated in FIG. 7, a needle assembly 700 having a sliding lever 752 is axially displaced distally toward the distal end 712 from the proximal end 711 of the housing 710. As shown, the sliding lever 752 is in a retracted position whereby the blunt distal tip of the elongate stylet portion 732 extends beyond the distal tip of the elongate needle portion 722. In one embodiment, the elongate stylet portion 732 extends through the elongate needle portion 722. Moreover, the elongate stylet portion 732 may include a hollow cavity along with a hole formed between inner and outer walls of the elongate stylet portion 732 so as to introduce anesthetic to a region of interest.

In another embodiment according to FIG. 7, an indicator or marker 770 may be provided on or in the needle housing 710 to allow a user to visually discern whether the needle portion 722 is in a retracted or extended position. The indicator may be color driven, such as for example, green and red markers. Other indicators, such as for example, letters, numbers, symbols or characters may also be employed as envisaged within the level of ordinary skill in the art. One marker, for example, may suggest that the needle assembly is safe for moving under the skin attributed to the blunt distal tip of the stylet extending beyond the sharp distal tip of the needle. Another marker, for example, may suggest that the needle assembly has a shart distal tip extending beyond the blunt distal tip of the stylet. An indicator similar to indicator 770 may be implemented in further embodiments as shown and described above in FIGS. 1-6

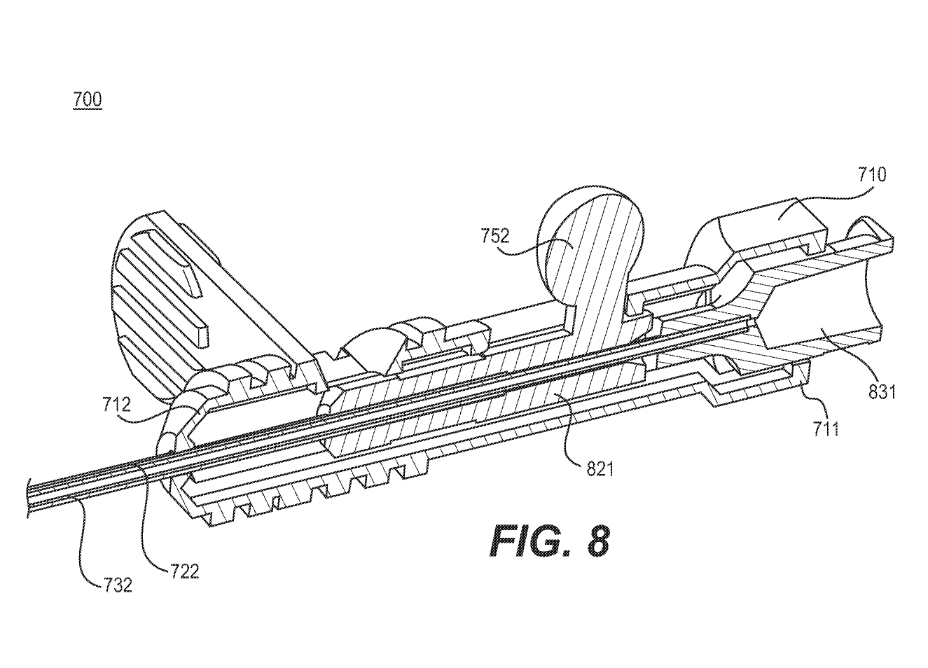

A cross-sectional view of the needle assembly 700 shown in FIG. 7 is illustrated in FIG. 8. A stylet hub 831 is coupled to a proximal end 711 of the housing 710. The stylet hub 831 is coupled to a elongate stylet portion 732. The elongate stylet portion includes a blunt distal end (not shown). The elongate stylet portion 732 is fixably attached to the stylet hub 831. There is a needle hub 821 disposed around the stylet hub 831. The elongate stylet portion 732 is fixedly held with respect to the housing 710. Coupled to the needle hub 821 is a sliding lever 752. The needle hub 821 and sliding lever 752 may be formed as a single unitary structure. The sliding lever is capable of axially displacing the needle hub 821 and an elongate needle member 722 having a sharp distal tip (not shown) extending therefrom from a retracted position to an extended position and vice versa. In a further embodiment, a spring (not shown) may be located in the housing 710. The spring may be a compression spring that is generally relaxed, e.g., passive state, when the blunt distal tip of the stylet is extending beyond the distal tip of the needle. The compression spring is generally compressed, or being compressed, when the distal tip of the needle extends beyond the blunt distal tip of the stylet.

According to yet a further aspect of the invention, there is described a method of improving navigation of the safety needle device toward a nerve. In one embodiment, the safety needle device includes an elongate needle portion having a distal tip. The device may also include an elongate stylet portion having a distal tip. In a further embodiment, the safety needle device may include features discussed earlier in the disclosure. In the method, the safety needle device is inserted via the distal end through a surface of a patient toward a nerve. The surface of the patient generally includes skin or tissue. The elongate needle portion is then moved in relation to the elongate stylet portion. Preferably, the elongate stylet portion remains fixed. This portion may be attached to an inner wall of the housing as discussed above. The needle portion moves from an extended position where its distal tip extends beyond the distal tip of the stylet to a retracted position where the distal tip of the stylet extends beyond the distal tip of the needle. In a further embodiment, the safety needle being configured in the retracted position is advanced toward the nerve. Since the distal tip of the elongate stylet portion is extended beyond the distal tip of the elongate needle portion, the risk of puncturing a nerve is reduced or eliminated.

Moreover, the elongate needle portion is locked in the retracted position before the safety needle device is advanced further under the surface of the skin. The locking feature is a safety measure to ensure that the distal tip does not move and possibly puncture a nerve.

In a further embodiment, the moving step may include applying a force to a locking handle. The locking handle and its orientation with respect to the safety needle device housing was described in detail above. For example, in relation to FIGS. 5 and 6, the force is applied to the locking handle 550 causing a portion thereof to translate along a longitudinal axis of the safety needle device 100. Preferably, normal force is applied to the locking handle 550. More preferably, normal force is applied to a stopping member 555 which is depressed toward an outer wall 510a of the housing 110. By so doing, a terminal portion 551 of the locking handle 550 moves out of contact or phase with a surface of a protrusion member 115. Once the terminal portion 551 moves out of contact with a surface of the protrusion member 115, the locking handle 550 moves or slides along a longitudinal axis toward a proximal end 111 of the housing 110.

As discussed above, the protrusion member 115 extends from an outer wall 510a of the housing 110. Moreover, the protrusion member 115 is located adjacent the proximal end 111 of the housing 110. The protrusion member 115 also includes a slot 615b formed therein. The slot 615b may be a recesses formed therein having a specific length and width to accommodate a section of the locking handle 550 including at least the terminal portion 551. Moreover, the slot 615b may accommodate a section of the locking handle 550 located between the proximal terminal portion 551 and the stopping member 555.

In another embodiment, the moving step causes a spring in the safety needle device to be displaced. Namely, the spring moves between two extreme states—a contracted state and an expanded state. The contracted state of the spring occurs when the elongate needle portion is in an extended position. The expanded state of the spring occurs when the elongate needle portion is in a retracted position.

In even a further embodiment, the method of navigating toward a nerve includes a step of injecting anesthesia through the elongate stylet portion. Preferably, the anesthesia is delivered when the elongate needle portion is in a retracted state. Thus, the distal tip of the elongate stylet portion extends beyond the distal tip of the elongate needle portion. Preferably, the elongate stylet portion extends through the elongate needle portion.

According to yet another aspect of the invention, a system of administering anesthesia to an area located between a substrate is described. The substrate may include an outer and inner wall. In one embodiment, the substrate may include a body, including but not limited to, humans, animals, fish, and artificial intelligence. Moreover, the substate may include various species of plant life. The system also includes a safety needle device including an elongate needle portion having a distal tip and an elongate stylet portion having a distal tip. The elongate needle portion is moved from an extended position where its distal tip extends beyond the distal tip of the stylet to a retracted position where the distal tip of the stylet extends beyond the distal tip of the needle. Preferably, the elongate stylet portion remains in a fixed state in relation to the elongate needle portion. For example, the elongate stylet portion may be fixed to an inner wall of the housing. The device is locked once the elongate needle portion moves into the retracted position. In addition, the system includes anesthesia that is supplied through the elongate stylet portion to an area between the outer and inner wall of the substrate In one embodiment, the elongate stylet portion includes a lumen for dispensing anesthesia to the area.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A safety needle device comprising:
a housing including a proximal end and a distal end extending along a longitudinal axis of the housing;
a needle partially disposed located in the housing, having an elongate needle portion with a distal tip, the elongate needle portion extending distally from the distal end of the housing;
a stylet fixed to the housing, the stylet including an elongate portion with a blunt distal tip extending distally out of the distal end of the housing;
a biasing member coupled to the housing and needle; and
a sliding member coupled to the needle, configured to the move the needle between an extended and a retracted position, such that the distal tip of the needle extends beyond the distal tip of the stylet in the extended position and the distal tip of the stylet extends beyond the distal tip of the needle in the retracted position, the needle being slidably disposed relative to the stylet and housing, the biasing member acting to bias the needle towards the retracted position.

2. The device according to claim 1, further comprising:
a locking mechanism coupled to the needle, configured to lock the needle in both the extended and retracted positions.

3. The device according to 1, wherein the locking mechanism is substantially located outside of the housing.

4. The device according to claim 1, wherein the locking mechanism includes a locking handle having a stopping member extending perpendicular to the longitudinal axis of the housing.

5. The device according to claim 4, wherein the housing includes a protrusion member located adjacent to the proximal end of the housing and extending perpendicular to the longitudinal axis of the housing.

6. The device according to claim 5, wherein the protrusion member defines a slot formed therein configured to receive a proximal terminal portion of the locking handle in the retracted position, the protrusion member further abutting against the stopping member in the retracted position to limit proximal movement of the locking handle.

7. The device according to claim 5, wherein the protrusion member includes a first surface configured to support the locking handle in the extended position.

8. The device according to claim 6, wherein the proximal terminal portion includes a surface substantially perpendicular to the longitudinal axis of the housing configured to abut against the protrusion member in the extended position.

9. The device according to claim 1, wherein the distal tip of the needle is substantially straight.

10. The device according to claim 1, wherein the elongate stylet portion extends through the elongate needle portion.

11. The device according to claim 1, wherein the elongate stylet portion includes a lumen for dispensing anesthesia.

12. The device according to claim 1, wherein the biasing member is a compression spring located in the housing between a needle hub at the proximal end of the needle and a distal end of the housing.

13. A method of navigating a safety needle device toward a point comprising:
providing the safety needle device including an elongate needle portion having a distal tip and an elongate stylet portion having a blunt distal tip;
introducing the distal tip of the needle portion through a surface;
moving the elongate needle portion after the introducing step from an extended position where the distal tip of the needle portion extends beyond the distal tip of the stylet portion to a retracted position where the distal tip of the stylet portion extends beyond the distal tip of the needle portion; and
advancing the safety needle while in the retracted position toward the point.

14. The method of claim 13, further comprising:
locking the elongate needle portion in the retracted position before the advancing step.

15. The method according to claim 13, further comprising:
locking the elongate needle portion in the extended position before the inserting step.

16. The method according to claim 13, wherein the step of moving to the retracted position includes applying a force to a locking mechanism coupled to the elongate needle portion causing a section of the locking mechanism to be displaced along a longitudinal axis of the safety needle device.

17. The method according to claim 13, further comprising:
extending the distal tip of the elongate needle portion past the blunt distal tip of the stylet to the extended position before the introducing step.
